(19) United States
(12) Patent Application Publication
(10) Pub. No.: US 2017/0112648 A1
(43) Pub. Date: Apr. 27, 2017

(54) MEDICAL INSTRUMENT DEPLOYER

(71) Applicant: Cook Medical Technologies LLC, Bloomington, IN (US)

(72) Inventor: Ryan Wiant, Toledo, OH (US)

(21) Appl. No.: 15/293,760

(22) Filed: Oct. 14, 2016

Related U.S. Application Data
(60) Provisional application No. 62/244,939, filed on Oct. 22, 2015.

(51) Int. Cl. A61F 2/966; A61F 2/01

(52) U.S. Cl. CPC A61F 2/966; A61F 2/01; A61F 2002/9665; A61F 2002/011; A61F 2002/9517

(57) ABSTRACT

The present disclosure provides for a medical instrument deployer having a barrier element such that the barrier element may move between a closed state, where the medical instrument is contained, and an open state, where the medical instrument is deployed within a body. Moving the barrier element between the closed state and the open state may involve rotating the barrier element around the circumference of the deployer.

### CROSS-REFERENCE TO RELATED APPLICATIONS

This application claims the benefit of priority under 35 U.S.C. §119(a) to U.S. Provisional Application No. 62/244,939, filed Oct. 22, 2015 all of which are hereby incorporated by reference in their entirety.

### BACKGROUND

1. Field of the Invention

The present disclosure relates to medical devices. More particularly, the disclosure relates to a device to deploy a medical implant or instrument into a body or body vessel.

2. Background

Medical instruments, such as stents and filters, often need to be implanted into a body portion, such as in a body vessel. These devices are often delivered or implanted through delivery devices. However, current delivery devices may result in a non-uniform deployment of the medical instrument, such as a non-uniform or asymmetrical implant of a stent along a body vessel. As such, there is a need for further delivery devices that can provide uniform deployment into the body.

### BRIEF SUMMARY OF THE INVENTION

The invention may include any of the following embodiments in various combinations and may also include any other aspect described below in the written description or in the attached drawings. In one embodiment, the device has an outer sheath and a barrier element. The outer sheath has a proximal end, a distal end, and an outer wall extending from the proximal end to the distal end along a longitudinal axis, the outer sheath having a first lumen formed therethrough. The outer wall also has an opening.

The barrier element is moveably disposed about the opening in a circumferential direction to move the device between an open state and a closed state, the device being in the closed state when the barrier element is disposed at the opening, the device being in the open state when the barrier element is disposed away from the opening.

The device may further have an inner sheath having a first end extending along the longitudinal axis to a second end and a second lumen formed therethrough, the inner sheath being slidably received within the first lumen, the inner sheath having a second portion between the first and second ends being closer to the second end than the first end. The inner sheath may cooperate with an implant, being disposed about the second portion and cooperating with the barrier element such that the implant moves through the opening and into the body in the open state.

The outer wall may have a handle track proximal the opening. In addition, the barrier element may have a base and a barrier wall extending distally from the base along the longitudinal axis. The device may further include an outer rod disposed in the first lumen and having a first handle extending therefrom, the first handle being disposed through the handle track, the outer rod distally extending and connected to the base such that longitudinal movement of the outer rod allows movement of the barrier element about the opening in the circumferential direction thereby moving the device between the open and closed states.

The barrier element may be movable in a helical pattern along the longitudinal axis, the barrier element being rotational in the circumferential direction about 180 degrees.

To move the barrier element, the outer wall may have an inner surface forming a first coupling extending helically along the longitudinal axis, the barrier element having an exterior forming a second coupling being slidably connected to the first coupling such that the barrier element moves helically along the longitudinal axis.

The device may further comprise a wire guide disposed in the second lumen for positioning the device in the body. The barrier base may form an aperture to accommodate the wire guide. The aperture may be disposed about the inner sheath, the barrier wall being arcuate.

The inner sheath may have an atraumatic tip at the second end, the atraumatic tip being removably coupled to the second portion, the atraumatic tip forming the second lumen therethrough and a third coupling, barrier element comprising a base and a barrier wall extending distally from the base along the longitudinal axis, the barrier wall being arcuate, the barrier element having an exterior forming a second coupling slidably connected to the third coupling, the atraumatic tip comprising a wall track formed to slidably receive the arcuate barrier wall.

The device may have a second handle being proximal the proximal end and attached to an inner rod extending distally from the second handle to the first end such that the second handle is operable to longitudinally move the inner sheath. The outer rod is disposed about the inner rod. The opening may be formed along the longitudinal axis and sized and shaped to accommodate expansion of the implant.

The device has as one possible advantage that the stent may be deployed uniformly, or all at once along the full length of the stent. This may avoid previous deployment problems where the stent was non-uniformly deployed in the vessel (e.g. by a wire guide pressed against the vessel wall during deployment). The present disclosure may be better understood by referencing the accompanying Figures.

### BRIEF DESCRIPTION OF THE DRAWINGS

FIGS. 1A-C depict break-away views of a medical device in accordance with one embodiment of the present invention.

FIG. 2 depicts a partial side view of the medical device of FIG. 1A.

FIGS. 3A-B depict partial side views of a barrier element of the medical device of FIG. 1A.

FIGS. 4A-C depict partial views of a first coupling and a second coupling of the medical device of FIG. 1A.

FIGS. 5A-B depict views of a tip of the medical device of FIG. 1A.

FIG. 6 depicts a partial break-away view of the medical device of FIGS. 1A-B.

FIGS. 7A-B depict partial views of the first handle of the medical device of FIG. 1B.

FIGS. 8A-C depict partial views of implant delivery with the medical device of FIGS. 1A-B.

### DETAILED DESCRIPTION

The present disclosure will now be described more fully with reference to the accompanying Figures, which show preferred embodiments. The accompanying Figures are provided for general understanding of the structure of various embodiments. However, this disclosure may be embodied in many different forms. These Figures should not be construed as limiting and they are not necessarily to scale.

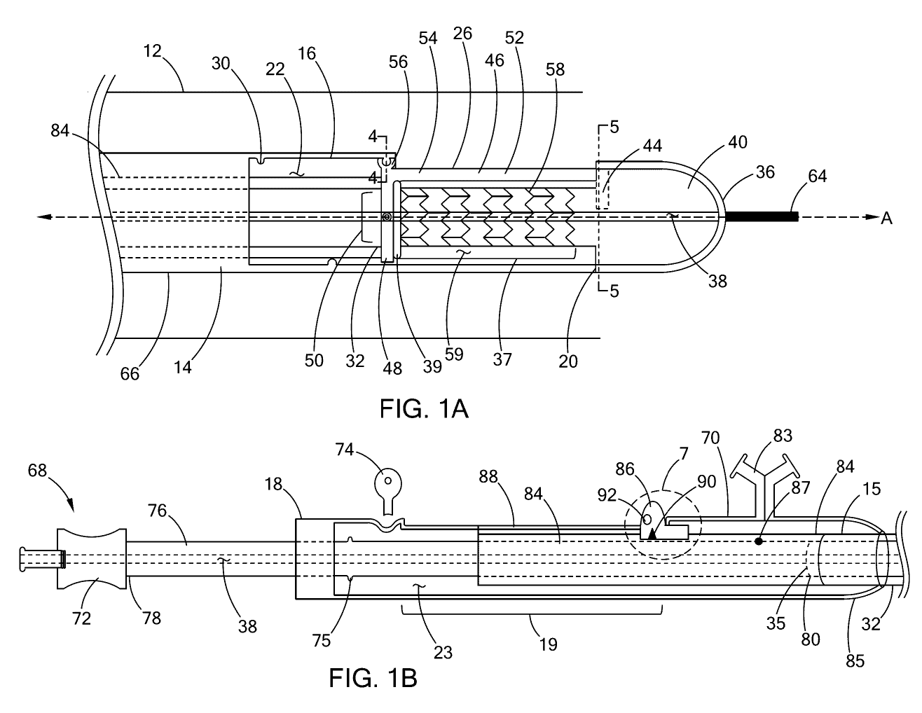

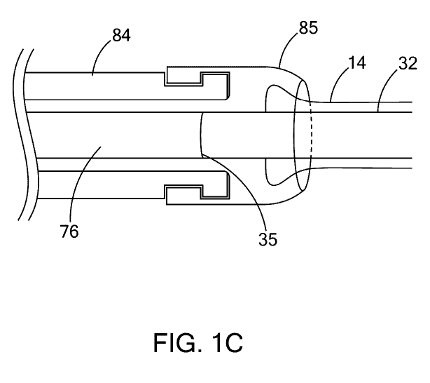

FIGS. 1A-C depict break-away views of the device. In FIG. 1A-B, the outer sheath 14 is placed within a body vessel 12 having a vessel wall. The outer sheath has an outer wall 66 extending from a proximal end 15 to a distal end 20, along a longitudinal axis A. The outer sheath 14 has a first lumen 22 formed therethrough. The outer wall 66 also has an opening 26 formed adjacent to the distal end 20, as seen in FIG. 1A.

The device also has the barrier element 46 movably disposed about the opening 26 in the circumferential direction (i.e. around the device circumference) to move the device between an open state and a closed state. The closed state being shown in FIG. 1A. The device is in the closed state when the barrier element 46 is disposed about the opening 26. The device is in the open state when the barrier element 46 is disposed away from the opening 26. As depicted in FIG. 1A, the barrier element 46 may be slightly recessed from the outer wall 66 within the opening 26. This recess may accommodate movement of the barrier element 46, as will be discussed further below.

The device further has an inner sheath 32. The inner sheath 32 has a first end 35 extending along the longitudinal axis A to a second end 36, with a second lumen 38 formed therethrough. The inner sheath 32 is slidably disposed within the first lumen 22. The inner sheath 32 has a second portion 37 between the first and second ends (35, 36) and being closer to the second end 36 than the first end 35.

Additionally, the inner sheath 32 may be slidably disposed through the barrier element 46. To accommodate this, the barrier element 46 may have an aperture 50 at the location of the bracket. In this side view, the aperture 50 is not visible. However, the bracket indicates where the aperture would be. The aperture may be sized to accommodate the inner sheath 32. The barrier element may move independently of the inner sheath 32 and accommodate the inner sheath's circumference such that the barrier element rotates or moves around the inner sheath 32. In some cases, the inner sheath may be proportionally smaller than shown in FIGS. 1A-C. Here, it may be enlarged to show detail.

The second portion 37 holds or accommodates an implant 58 within cavity 59. The implant 58 may be disposed about the second portion 37. The implant 58 cooperates with the barrier element 46 such that the implant 58 moves through the opening 26 and into the body in the open state. The opening 26 is formed along the longitudinal axis A and is sized and shaped to accommodate expansion of the implant 58. The inner sheath 32 may form an extrusion 39 at a proximal section. Extrusion 39, located adjacent to the barrier element 46, helps to align the implant 58 and holds it in place such that it can expand into the body vessel 12.

It will be understood to one of skill in the art that the implant 58 could be any medical instrument delivered to any place in the body. As shown in FIG. 1A, the implant 58 is a stent for implantation into a body vessel 12. The implant 58 could also be a filter, a tool for temporary use, and the like.

The implant 58 may be delivered to the body vessel by operation of the barrier element 46. The barrier element 46 may have a base 48 and a barrier wall 52 extending distally from the base 48, along the longitudinal axis A. The barrier wall 52 may be arcuate to fit inside the outer sheath 14 and around the tubular implant 58.

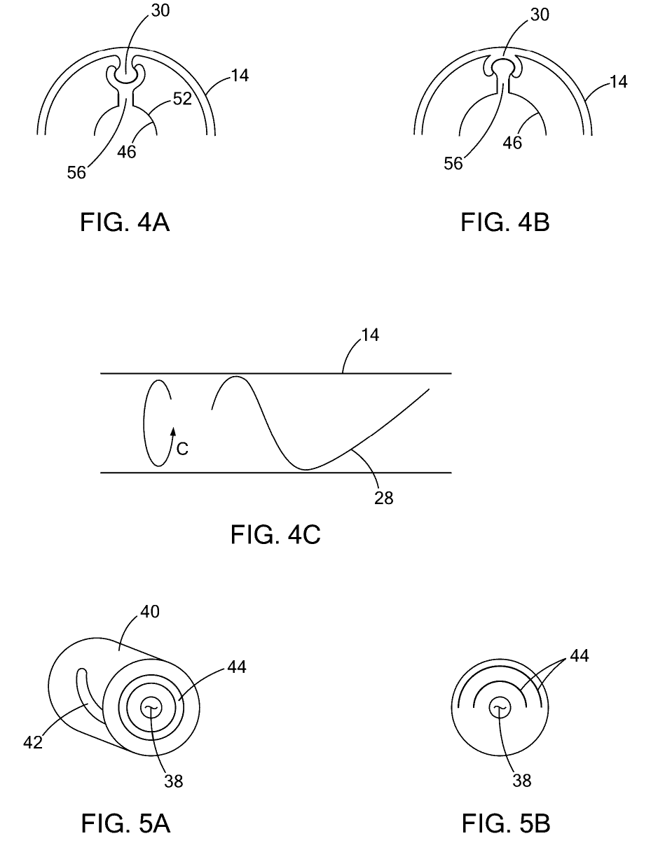

The barrier element 46 is movable in a helical pattern along the longitudinal axis A. It may be rotated about the circumference about 180°. To accommodate the barrier element 46, the outer wall 66 has an inner surface 16 forming a first coupling 30 extending helically along the longitudinal axis A. The barrier element 46 has an exterior 54 forming a second coupling 56 being slidably connected to the first coupling 30 such that the barrier element 46 moves helically along the longitudinal axis A. In FIG. 1A, the first and second couplings (30, 46) are shown in a break-away view. Further details of the first coupling will be described with FIGS. 4A-C.

In order to operate the barrier element 46 along with the inner surface 16, the proximal part of the device is equipped with a delivery system 68. As part of the delivery system 68, a second handle 70 forms a handle track 88 proximal the opening 26, the handle track 88 being disposed at a segment 19 of the second handle 70. The second handle 70 forms a third lumen 23 and is coaxial with the outer sheath 14. The handle has a proximal handle end 18.

Further, the delivery system 68 includes an outer rod 84 disposed within the third lumen 23. The outer rod 84 has a first handle 86 extending therefrom and being disposed through the handle track 88. The outer rod 84 distally extends and connects to the proximal end 15. Longitudinal movement of the outer rod 84 moves the outer sheath 14 and allows or creates movement of the barrier element 46 about the opening 26 in the circumferential direction, thereby moving the device between the open and closed state.

In one embodiment, as shown in FIG. 1B, the handle track 88 will be rectangular to allow only translational movement of the first handle 86. The user's translational movement may cause translational and rotational movement at the distal part of the device, moving the barrier element 46. However the handle track 88 could be formed to allow rotational and translational movement of the first handle 86, such that the handle track is helical to mimic the helical first coupling 30. In this alternative embodiment, translational and rotational movement by the user at the proximal end will cause translational and rotational movement by the barrier element 46 at the distal end.

The first handle 86 also has a first lock 90 and a first indent 92 to lock or immobilize the first handle 86 during storage. Details of these elements will be discussed further with FIGS. 7A-B.

Returning to FIG. 1A, the inner sheath 32 also has an atraumatic tip 40 at the second end 36. The atraumatic tip 40 may be removably coupled to the distal end 20 such that the user may withdraw the outer sheath 14 from the body vessel 12, but the atraumatic tip 40 and the inner sheath 32 will remain in place in the body vessel 12.

The atraumatic tip 40 also forms the second lumen 38 therethrough and a third coupling (further discussed with FIGS. 5A-B). The barrier element 46 has the exterior 54 that may form multiple second couplings 56, at least one may be slidably connected to the third coupling 42 as well as the first coupling 30. The atraumatic tip 40 may also have a wall track 44 formed to slidably receive the arcuate barrier wall 52.

Operation of the inner sheath 32 will now be discussed. As part of the delivery system 68, the device has a knob 72 disposed proximal the proximal handle end 18 and attached to an inner rod 76. The inner rod 76 may extend distally from the knob 72 to the first end 35 such that the knob 72 is operable to longitudinally move the inner sheath 32. The user may grasp or hold knob 72 to operate the inner sheath 32. The inner rod 76 may have a first rod end 78 extending to a second rod end 80 attached to the first end. The inner rod 76 may also form the second lumen 38. The user may manipulate the inner rod 76 to adjust or manipulate the inner sheath 32.

During storage of the device, the inner rod 76 may be locked in place through a rod lock 74 shown in FIG. 1B. The rod lock 74 may fit around a projection 75 in the inner rod 76 such that when the rod lock 74 engages projection 75 to immobilize or lock the inner rod 76 and inner sheath 32 relative to the other elements of the device. Inner rod 76 is immobilized along the longitudinal axis A. Before operation, the user will disengage the rod lock 74 from the inner rod 76 in order to operate the device in the body.

The inner rod 76 is disposed through the outer rod 84 such that the outer rod 84 is disposed about the inner rod 76. The two rods may not be attached, but they may operate adjacent to each other to move different parts of the device.

The second handle 70 also may be associated with a port 83, which can flush a fluid between the outer and inner rods, and between the outer and inner sheaths. Such port 83 may be able to flush a fluid when it aligns with hole 87. Hole 87 may provide fluid communication between the outer and inner rods and between the outer and inner sheaths.

The device may also have a cap 85 positioned at or distal to the first end 35, at a distal end of the second handle 70. The cap 85 may allow the outer and inner sheaths (14, 32) to extend from the delivery system 68 and through the cap 85 to the target body vessel location. FIG. 1C shows a modified form of the cap 85, in which the cap 85 is a spin cap. The spin cap 85 may be able to spin with the barrier element as the first handle 86 is pulled proximally. In this embodiment, the outer sheath 14 may twist, preventing possible kinks or damage to the device. In any embodiment, the outer sheath 14 may be flared and glued into the cap 85. The spin cap, or any other twistable part of the device, may be lubricated to facilitate movement.

The device may also be usable with a wire guide 64. The wire guide 64 may be disposed in the second lumen 38 for positioning the device in the body. The wire guide allows the user to track the device to the appropriate location in the body.

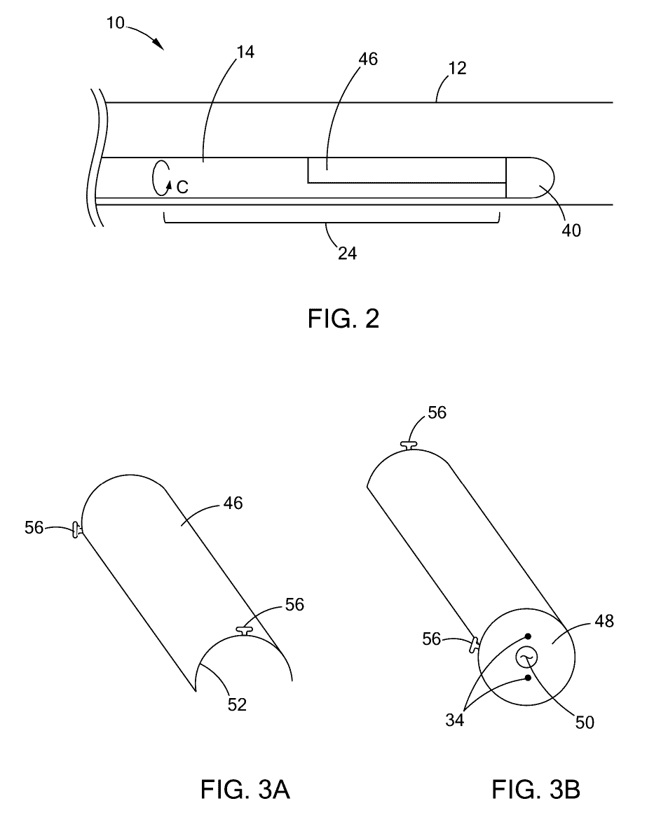

Turning to FIG. 2, the device is shown in the body 10 with body vessel 12, in an environmental view. The outer sheath 14 extends distally to a distal portion 24 of the device, housing the implant. The barrier element 46 is closed such that the implant is contained within the device. FIG. 2 further shows the atraumatic tip 40 at the distal end. The device has a circumferential direction C, in which it operates or moves.

FIGS. 3A-B illustrate the barrier element 46, shown separate from the device. The barrier element 46 is shown arcuate. FIG. 3A shows a front end view of the barrier element 46. FIG. 3B shows a back end view of the barrier element 46. In FIG. 3A, the barrier element 46 has a plurality of second couplings 56 that may connect to the first and third couplings of the device. In this view, a distal second coupling 56 is disposed on the front end and a proximal second coupling 56 is disposed on the back end, rotated 90° from the distal second coupling 56. One skilled in this art will understand that additional second couplings may provide further stability for the barrier element as it moves.

FIG. 3B shows further details of the base 48, forming aperture 50. The aperture 50 may be sized and shaped to accommodate the inner sheath 32 and the wire guide 64. The base 48 may further comprise attachment points 34 for the outer sheath 14. In the case where translational movement of the outer rod creates translational and rotational movement of the barrier element 46, the attachment points 34 may be slidable tracks such that the base 48 may rotate independently of the outer rod, while the outer rod is attached to the outer sheath 14. Alternatively, the spin cap 85 (discussed with FIG. 1C) may create this independent rotation between the outer rod and the outer sheath 14/base 48 pair.

In the case where the outer rod has rotational and translational movement (e.g. helical movement), the attachment points 34 may be glued or welded such that the outer rod does not move independently from the outer sheath 14 and the barrier element. Such mechanical connection will be understood to one of skill in this art. In this case, there may be no need for a spin cap.

FIGS. 4A-C show further details of the couplings (depicted in Line 4-4 of FIG. 1A). In FIG. 4A, the outer sheath 14 has a first coupling 30 shown in this end view as a protrusion that would fit inside of the second coupling 56 of the barrier element 46. Here, the barrier element is depicted having an arcuate barrier wall 52.

FIG. 4B shows the opposite fit, where the first coupling 30 forms an opening to fit the second coupling 56, which is shown as a protrusion. Although FIGS. 4A-B are end views, it will be understood that the first coupling 30 would extend along the longitudinal axis, forming a type of track for the second coupling 56. FIG. 4C shows how the first coupling 30 would extend in a helical pattern 28 along the longitudinal axis to rotate the barrier element about 180° in the circumferential direction C.

FIGS. 5A-B show further details of the atraumatic tip 40 (depicted in Line 5-5 of FIG. 1A). FIG. 5A is a side view of the atraumatic tip 40 and FIG. 5B is an end view of the atraumatic tip 40. The atraumatic tip will form the second lumen 38 to house the wire guide. In addition, the atraumatic tip will have a wall track 44, which may be circular as shown in FIG. 5A. Alternatively, the wall track 44 may be semicircular as shown in FIG. 5B. In either case, the wall track 44 houses the barrier wall. If it is the case that the barrier wall extends into the atraumatic tip 40, it will slide or move within the wall track 44.

Additionally as shown in FIG. 5A, the atraumatic tip 40 may form a third coupling 42, similar to the first coupling, which may extend along an inner wall of the atraumatic tip 40. The third coupling 42 connects and/or coupled to the second coupling of the barrier element. If the barrier element has a second coupling at the front end, shown in FIGS. 3A-B, the second coupling could couple to the third coupling 42. This may provide additional stability for the barrier element during movement.

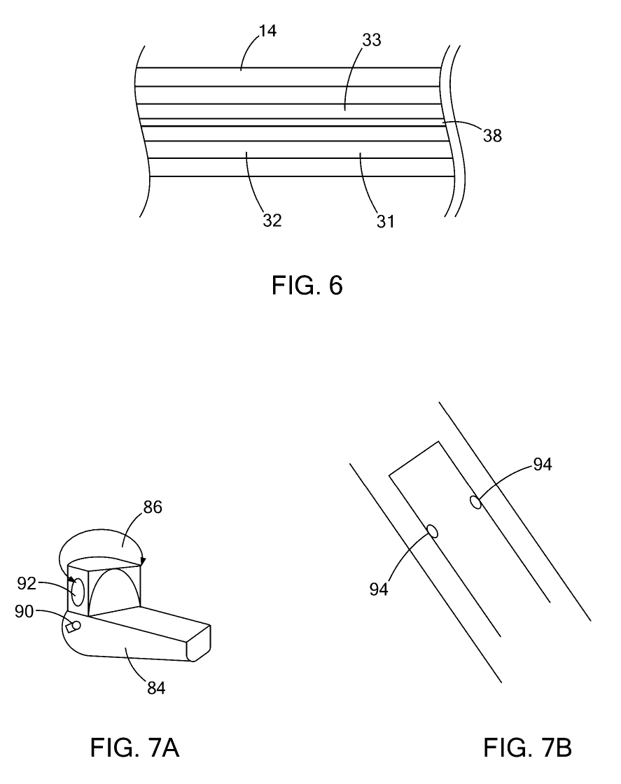

FIG. 6 shows a partial view of the device at a middle portion. In this side view, the outer sheath 14 is disposed about the inner sheath 32. The inner sheath 32 has an outer surface 31 and an inner surface 33. The wire guide lumen 38 runs along the inner surface 33.

The materials to form the outer sheath and the inner sheath would be any materials with sufficient stiffness and flexibility to track to a location with a body. For example, a polymer material could be employed that has a sufficient durometer to perform the movements discussed herein and be manipulated by the user from outside the body, but also sufficient flexibility to move through the body.

FIGS. 7A-B show views of the first handle 86 extending from the outer rod 84 (depicted with Circle 7 in FIG. 1B). The first handle 86 would also have a first lock 90 and a first indent 92 to lock the first handle in place until the user desires to move the first handle 86. The first indent 92 would be removably engaged with the first catch 94 in the second handle (shown in FIG. 7B) such that the device may be delivered with the first indent 92 engaged with the first catch 94. The user may squeeze the first handle 86 to disengage the first catch 94 from the first indent 92 to move the first handle 86 about and/or along the longitudinal axis. Likewise, the first lock 90 could fit in an indent within the second handle and be disengaged from the outer sheath upon operation or squeezing by the user.

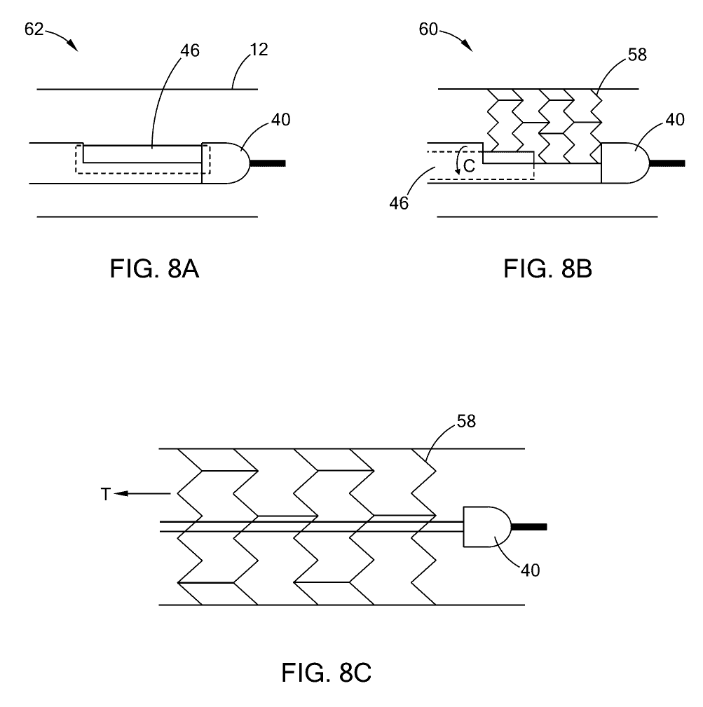

FIGS. 8A-C shows a method of operation of the device. In FIG. 8A, the device is in the closed state 62 within the body vessel 12. The barrier element 46 and the atraumatic tip have been tracked or positioned to the appropriate location in the body. In FIG. 8B, the user rotates the barrier element 46 along the circumferential direction C and translocates the barrier element 46 such that the implant 58 may expand out of the opening and engage the vessel wall. In this view, the device has been moved to the open state 60.

In FIG. 8C, the user has withdrawn the outer sheath in the direction of arrow T towards the proximal end. This allows the implant 58 to fully expand to the wall of the body vessel 12. The inner sheath with the atraumatic tip 40 remains in place. Now the user may withdraw the inner sheath proximally as well to leave the implant in place, being fully expanded.

It should be understood that the foregoing relates to exemplary embodiments of the disclosure and that modifications may be made without departing from the spirit and scope of the disclosure as set forth in the following claims. While the disclosure has been described with respect to certain embodiments it will be appreciated that modifications and changes may be made by those skilled in the art without departing from the spirit of the disclosure.

1. A device for use in a body, the device comprising:
an outer sheath having a proximal end, a distal end, and an outer wall extending from the proximal end to the distal end along a longitudinal axis, the outer sheath having a first lumen formed therethrough, the outer wall having an opening; and
a barrier element moveably disposed about the opening in a circumferential direction to move the device between an open state and a closed state, the device being in the closed state when the barrier element is disposed at the opening, the device being in the open state when the barrier element is disposed away from the opening.

2. The device of claim 1 further comprising an inner sheath having a first end extending along the longitudinal axis to a second end and a second lumen formed therethrough, the inner sheath being slidably received within the first lumen, the inner sheath having a second portion between the first and second ends being closer to the second end than the first end.

3. The device of claim 1 further comprising an implant disposed about the second portion and cooperating with the barrier element such that the implant moves through the opening and into the body in the open state.

4. The device of claim 1 further comprising a second handle proximal the proximal end and having a third lumen and a handle track formed therethrough, the second handle being coaxial with the outer sheath.

5. The device of claim 4 wherein the barrier element comprises a base and a barrier wall extending distally from the base along the longitudinal axis.

6. The device of claim 5 further comprising an outer rod disposed in the third lumen and having a first handle extending therefrom, the first handle being disposed through the handle track, the outer rod distally extending and connected to the proximal end such that longitudinal movement of the outer rod allows movement of the barrier element about the opening in the circumferential direction thereby moving the device between the open and closed states.

7. The device of claim 1 wherein the barrier element is movable in a helical pattern along the longitudinal axis, the barrier element being rotational in the circumferential direction about 180 degrees.

8. The device of claim 1 wherein the outer wall has an inner surface forming a first coupling extending helically along the longitudinal axis, the barrier element having an exterior forming a second coupling being slidably connected to the first coupling such that the barrier element moves helically along the longitudinal axis.

9. The device of claim 2 wherein the device further comprises a wire guide disposed in the second lumen for positioning the device in the body.

10. The device of claim 2 wherein the barrier element comprises a base and a barrier wall extending distally from the base along the longitudinal axis, the base forms an aperture disposed about the inner sheath, the barrier wall being arcuate.

11. The device of claim 2 wherein the inner sheath comprises an atraumatic tip at the second end, the atraumatic tip being removably coupled to the second portion, the atraumatic tip forming the second lumen therethrough and a third coupling, barrier element comprising a base and a barrier wall extending distally from the base along the longitudinal axis, the barrier wall being arcuate, the barrier element having an exterior forming a second coupling slidably connected to the third coupling, the atraumatic tip comprising a wall track formed to slidably receive the arcuate barrier wall.

12. The device of claim 6 further comprising a knob disposed proximal the second handle and attached to an inner rod such that the inner rod extends distally from the knob to the first end such that the knob is operable to longitudinally move the inner sheath.

13. The device of claim 12 wherein the outer rod is disposed about the inner rod.

14. The device of claim 3 wherein the opening is formed along the longitudinal axis and sized and shaped to accommodate expansion of the implant.

15. A device for use in a body, the device comprising:
an outer sheath having a proximal end, a distal end, and an outer wall extending from the proximal end to the distal end along a longitudinal axis, the outer sheath having a first lumen formed therethrough, the outer wall having an opening;
a barrier element moveably disposed about the opening in a circumferential direction to move the device between an open state and a closed state, the device being in the closed state when the barrier element is disposed at the opening, the device being in the open state when the barrier element is disposed away from the opening;
an inner sheath having a first end extending along the longitudinal axis to a second end and a second lumen formed therethrough, the inner sheath being slidably received within the first lumen, the inner sheath having a second portion between the first and second ends; and
an implant disposed about the second portion and cooperating with the barrier element such that the implant moves through the opening and into the body in the open state.

16. The device of claim 15 further comprising a second handle proximal the proximal end and having a third lumen and a handle track formed therethrough, the second handle being coaxial with the outer inner sheath, and the barrier element comprises a base and a barrier wall extending distally from the base along the longitudinal axis, the device further comprising an outer rod disposed in the third lumen and having a first handle extending therefrom, the first handle being disposed through the handle track, the outer rod distally extending and connected to the proximal end such that longitudinal movement of the outer rod allows movement of the barrier element about the opening in the circumferential direction thereby moving the device between the open and closed states.

17. The device of claim 15 wherein the barrier element is movable in a helical pattern along the longitudinal axis, the barrier element being rotational in the circumferential direction about 180 degrees.

18. An apparatus for use in a body, the apparatus comprising:
an outer sheath having a proximal end, a distal end, and an outer wall extending from the proximal end to the distal end along a longitudinal axis, the outer sheath having a first lumen formed therethrough, the outer wall having an opening;
a barrier element moveably disposed about the opening in a circumferential direction to move the device between an open state and a closed state, the device being in the closed state when the barrier element is disposed at the opening, the device being in the open state when the barrier element is disposed away from the opening, the barrier element comprising a base and a barrier wall extending distally from the base along the longitudinal axis;
a second handle proximal the proximal end and having a third lumen and a handle track formed therethrough, the second handle being coaxial with the outer sheath; and
an outer rod disposed in the third lumen and having a first handle extending therefrom, the first handle being disposed through the handle track, the outer rod distally extending and connected to the proximal end such that longitudinal movement of the outer rod allows movement of the barrier element about the opening in the circumferential direction thereby moving the device between the open and closed states.

19. The device of claim 18 wherein the barrier element is movable in a helical pattern along the longitudinal axis, the barrier element being rotational in the circumferential direction about 180 degrees.

20. The device of claim 18 wherein the outer sheath has an inner wall forming a first coupling extending helically along the longitudinal axis, the barrier element having an outer wall forming a second coupling being slidably connected to the first coupling such that the barrier element moves helically along the longitudinal axis.
